(19) United States
(12) Patent Application Publication
(10) Pub. No.: US 2010/0120062 A1
(43) Pub. Date: May 13, 2010

(54) RHIZOME VIABILITY DETERMINATION METHOD

(76) Inventor: Richard Mathew Meade, Bristol (GB)

Correspondence Address: RENNER KENNER GREIVE BOBAK TAYLOR & WEBER, FIRST NATIONAL TOWER FOURTH FLOOR, 106 S. MAIN STREET, AKRON, OH, 44308, US

(21) Appl. No.: 12/531,357

(22) PCT Filed: Feb. 19, 2008
(86) PCT No.: PCT/GB2008/050106

§ 371 Date: Sep. 15, 2009

(30) Foreign Application Priority Data
Mar. 16, 2007 (GB) 0705069.3

(51) Int. Cl. G01N 33/53; C12Q 1/02

(52) U.S. Cl. 435/7.2; 435/29

(57) ABSTRACT

A method of determining the viability of a rhizome comprises the sequential steps of: (a) sampling material from the rhizome; (b) contacting material sampled in step (a) with an effective amount of a formulation comprising a first viability indicator; and (c) observing the action of the first viability indicator on the sampled rhizome material, to determine the viability of the rhizome. The method may further comprise one or more additional sequences involving the additional steps of: (d) contacting material sampled in step (a) with an effective amount of a formulation comprising a further viability indicator different to that used in step (b); and (e) observing the action of the further viability indicator on the sampled rhizome material, to determine the viability of the rhizome. In this way, an overall assessment of the viability of the rhizome can be made based on the action of a plurality of viability indicators, each relying on an interaction with different reagents in the plant cells.

This invention relates to a method for determining the viability of plant rhizomes. In particular, it relates to a method for determining the viability of rhizomes of pernicious weeds such as Japanese knotweed (Fallopia japonica) as part of a weed control programme. The method of the present invention has been developed for use in connection with Japanese knotweed, and will therefore be described herein with particular emphasis on this plant. It is envisaged however that the method of the present invention will be applicable to substantially all unwanted rhizomatous plants, and references herein to rhizomes in general and Japanese knotweed in particular should therefore be construed accordingly.

Invasive rhizomatous plant species such as Japanese knotweed can spread rapidly by vegetative reproduction, and in particular by the dispersal of rhizome fragments. Much dispersal of such invasive rhizomatous species has been caused by incorrect handling of soils, with rhizome fragments often being moved as general garden waste, or by movement of soils for construction projects. This has greatly aided the spread of Japanese knotweed in particular. Laboratory experiments have shown that a new Japanese knotweed plant can grow from a rhizome fragment as small as 0.22 g.

As a result of this spread, invasive rhizomatous plant species such as Japanese knotweed grow wild both in the countryside, where they can disrupt the biodiversity of the surrounding area, and in cities, where their roots can grow into joints and weaknesses in concrete structures causing damage and making the structures unsafe. The eradication of these species, especially from construction sites and the like, is therefore highly desirable. A method of controlling Japanese knotweed is described in the applicant's International Publication No. WO2006/103478.

In view of the ease with which invasive rhizomatous plant species such as Japanese knotweed spread, it is important to ascertain whether the rhizome system in an infested area has been completely killed, following treatment of that area with systemic herbicides, for example using the method described in WO2006/103478. In order to be able to guarantee that no re-growth will occur following a treatment cycle, it must be ascertained that the rhizomes of the plant are dead, rather than merely dormant.

Conventional methods for determining the viability of rhizomes are, however, unsatisfactory and unreliable. For example, the current method recommended by the Welsh Development Agency to assess the viability of a Japanese knotweed rhizome involves keeping a sample moist for four weeks and observing whether new shoots grow. There are two main problems associated with this method: firstly, the method takes an unsatisfactorily long time to deliver results; and secondly, the method is unreliable as the rhizomes can enter into a dormant state during the winter months. Thus, dormant rhizomes could be scored as dead according to the Welsh Development Agency re-growth assay when in fact they are still viable.

The present invention seeks to provide an accurate, reliable and quicker method of determining the viability of a rhizome.

According to the present invention there is provided a method of determining the viability of a rhizome, comprising the sequential steps of:

(a) sampling material from the rhizome;

(b) contacting material sampled in step (a) with an effective amount of a formulation comprising a first viability indicator; and

(c) observing the action of the first viability indicator on the sampled rhizome material, to determine the viability of the rhizome.

The method of the present invention optionally comprises at least one additional sequence of:

(d) contacting material sampled in step (a) with an effective amount of a formulation comprising a further viability indicator different to that used in step (b); and

(e) observing the action of the further viability indicator on the sampled rhizome material, to determine the viability of the rhizome.

The method optionally further comprises a plurality of such additional sequences (d) and (e), each such sequence utilising a different viability indicator.

Preferably, at least one viability indicator utilised in the method is a vital stain indicator. Most preferably each viability indicator utilised in the method is a vital stain indicator.

Vital stain indicators work by the vital stain being chemically converted inside the living cells of sampled rhizome material. The reaction product can then be detected, usually by fluorescence or by a change in colour, depending on the viability stain used. The presence of a specific reaction product shows that there are living cells in the rhizome. Conversely, if the specific reaction product for a selected vital stain is not detected, this indicates that the rhizome does not contain living cells and hence is not viable.

Each vital stain indicator is preferably selected from the group consisting of fluorescein diacetate, resazurin and tetrazolium chloride salts. Where a tetrazolium chloride salt is selected, 2,3,5-triphenyltetrazolium chloride is most preferred.

Fluorescein diacetate (also referred to as 3′,6′-diacetylfluorescein or FDA) provides a rapid, simple and sensitive test for determining the viability of rhizome samples. FDA diffuses into the cell through the cell membrane. Once inside the cell, the acetate groups on the FDA are cleaved off by esterase inside the cell. This hydrolysis reaction generates the more polar, fluorescent molecule, fluorescein, which can be detected by fluorescence microscopy. Fluorescein diffuses out of the cell at a slower rate than the FDA flows into the cell, and fluorescein therefore accumulates over time. In non-living cells, no hydrolysis reaction occurs due to the absence of functioning esterases. As such, fluorescein is not generated, and hence no fluorescence is seen in these cells.

Resazurin is a redox-active dye which is initially blue in colour and non-fluorescent. Resazurin is reduced to resorufin in living cells by electron-transfer reactions occurring during respiration. Resorufin is pink in colour and highly fluorescent—and as with FDA, the change in fluorescence can again be detected using fluorescence microscopy. Resazurin has previously been used to determine milk quality, and the viability and numbers of gram-positive bacteria, gram-negative bacteria, and fungi. It does not however appear previously to have been used in a viability assay context in relation to higher eukaryotes.

Viability assays using tetrazolium chloride salts, and in particular, 2,3,5-triphenyltetrazolium chloride (TTC), have been found to be particularly sensitive and quantitative—although not as quick to produce a result as the FDA or resazurin assays. When TTC diffuses into living cells, it accepts electrons liberated by the action of dehydrogenase enzymes on respiration substrates during aerobic respiration. The colourless TTC is thus reduced to formazan, a pink coloured compound, which stains the plant tissue red. This red stain can then be extracted, and measured quantitatively using a spectrophotometer. The intensity of the colour is proportional to the rate of respiration in the tissue, and can therefore be used to assess the viability of that tissue.

Where fluorescein diacetate is selected as a vital stain indicator, the method of the present invention preferably further includes a preliminary step of preparing a fluorescein diacetate stain formulation comprising substantially 0.4 g fluorescein diacetate in 100 ml acetone. The operation of contacting the sampled rhizome material from step (a) with the fluorescein diacetate stain formulation in step (b), or in one of steps (b) or (d) if step (d) is present, preferably involves incubating the sampled rhizome material with substantially 5 ml of fluorescein diacetate stain formulation at room temperature for substantially 20 minutes.

Where fluorescein diacetate is selected as a vital stain indicator, the observation of the action of the fluorescein diacetate on the sampled rhizome material in step (c), or in one of steps (c) or (e) if step (e) is present, is preferably effected by fluorescence microscopy.

Where resazurin is selected as a vital stain indicator, the method of the present invention preferably further includes a preliminary step of preparing a resazurin stain formulation as an aqueous solution having a concentration of substantially 9.95 μM. The operation of contacting the sampled rhizome material from (a) with the resazurin stain formulation in step (b), or in one of steps (b) or (d) if step (d) is present, preferably involves incubating the sampled rhizome material with substantially 10 ml of resazurin stain formulation at room temperature for at least 15 minutes.

Where resazurin is selected as a vital stain indicator, the observation of the action of the resazurin on the sampled rhizome material in step (c), or in one of steps (c) or (e) if step (e) is present, is preferably effected by fluorescence microscopy.

In addition to its use in laboratory analysis, the resazurin viability test can also be utilised as a crude on-site assay.

Where a tetrazolium chloride salt is selected as a vital stain indicator, the method of the present invention preferably further comprises a preliminary step of preparing a tetrazolium chloride salt stain formulation as an aqueous solution having a concentration of substantially 0.6% (w/v) tetrazolium chloride salt and a pH of substantially 7.4 in a phosphate buffer. The operation of contacting the sampled rhizome material from step (a) with the tetrazolium chloride salt stain formulation in step (b), or in one of steps (b) or (d) if step (d) is present, preferably involves incubating substantially 0.3 g of sampled rhizome material with substantially 3 ml of tetrazolium chloride salt stain formulation for substantially 24 hours at substantially 30° C.

Where a tetrazolium chloride salt is selected as a vital stain indicator, step (b), or one of steps (b) or (d) if step (d) is present, preferably further comprises the sub-steps of washing each sample three times with deionised water; and subjecting each said sample to an extraction process, using substantially 4 ml of 95% ethanol at substantially 85° C. for substantially 5 minutes; said extraction process being repeated four times, and the resultant extracts combined and made up to 25 ml with 95% ethanol. The observation of the action of the tetrazolium chloride salt on the sampled rhizome material in step (c), or in one of steps (c) or (e) if step (e) is present, is preferably effected by measuring the absorbance of the sample at substantially 490 nm using a spectrophotometer.

Preferably, the selected tetrazolium chloride salt is 2,3,5-triphenyl-tetrazolium chloride.

As the three viability assays outlined above rely on the interaction of each vital stain indicator with different reagents in the plant cells, the assays each complement the action of the others in providing an overall assessment of the viability of sampled rhizome material. It is therefore highly preferable that the method of the present invention comprises two additional sequences of steps (d) and (e), and that each of fluorescein diacetate, resazurin and 2,3,5-triphenyltetrazolium chloride is selected as a vital stain indicator.

The step (a) of sampling material from the rhizome preferably comprises sub-steps of washing the rhizome to remove dirt and any contaminants, and slicing the rhizome into sections of substantially 1-2 mm thickness. Most preferably, in step material is sampled from the rhizome at nodal regions thereof containing a meristem.

### EXAMPLES

In order that the present invention may be more clearly understood, preferred embodiments thereof will now be described in detail, though only by way of example, with reference to experimental observations and data.

The following Examples illustrate the preparation and testing of rhizome samples using each of three preferred viability indicators: fluorescein diacetate (FDA), resazurin, and 2,3,5-triphenyltetrazolium chloride; and the results of experiments to assess the viability of Japanese knotweed rhizomes, and hence the effectiveness of a Japanese knotweed treatment programme.

### Example I

Japanese knotweed rhizome samples were collected from six different sites in the United Kingdom—Bath, Bristol, Shipdham, Aldridge Perry Barr, Runcorn and Chester. The samples collected from Bath and Bristol were collected from living plants the morning of the test. The rhizomes were collected from under the crown of the plant at a depth of about 30 cm. The samples collected from Shipdham, Aldridge Perry Barr, Runcorn and Chester were collected from sites which had been treated with a specific anti-knotweed preparation. The knotweed was considered to have been eradicated from these sites, and the rhizome samples were therefore believed to be dead. The same samples had already been tested for viability using the Welsh Development Agency re-growth assay, and based on that test has been assumed to be dead. From each site, three rhizome samples having over eight nodes, and a diameter of between 8-15 mm were collected. The collected rhizomes were cut into separate nodal sections. These sections were then used either to perform culture experiments or vital stain assays. Generally, the nodes used for culture experiments were sampled between the nodes used for the different vital stain assays.

### Example II

Samples of rhizome for use in culture experiments, all over one gram in weight, were selected from the nodal sections cut in Example I. The order of the samples was randomized from sample to sample to ensure the results were not influenced by the node order.

500 ml of rhizome culture solution was made up from 1.525 g of Duchefa Gambourg B5 Medium mixed with 0.025 g of gentamycin (making a final gentamycin concentration of 50 μg/ml) and 0.1% sucrose. The mixture was then made up to 500 ml using deionised water, and autoclaved. The resultant solution was then left to cool and 0.02112 g of carbenicillin (making a final carbenicillin concentration of 100 μM) and 0.015 g of nystatin (making a final nystatin concentration of 150 U/ml) were then added by filter sterilisation.

To prepare the samples, the rhizomes were first thoroughly washed in warm water to remove dirt. The samples to be used for the growth culture assays were then cut using a razor blade.

The growth culture assays were carried out in a sealed Tupperware® container. This container was surface-sterilised before the start of the experiment. This was done by first rinsing the container with a 0.5% bleach solution followed by five rinses of autoclaved deionised water to wash away the bleach. Finally, the container was washed with a 70% ethanol solution, and dried to prepare the container for receiving the samples. Three layers of cellulose filter paper (Whatman number 3) were laid down in the container, with one layer of glass fibre filter paper (Whatman GF/A) on top. The samples were placed on top of the glass fibre filter paper and the culture solution was added to the container, to the depth of the filter paper. The samples were then left for 2 weeks at room temperature (approx. 21° C.) in natural summer light, and inspected periodically for growth.

### Example III

A fluorescein diacetate (FDA) stain formulation was prepared by dissolving 0.4 g FDA in 100 ml of acetone. 5 ml of this solution was then used for each sample tested.

To prepare the samples, the rhizomes collected in Example I were washed thoroughly to remove any soil or other contaminants. Thin hand-cut sections, 1-2 mm thick, were then taken from nodal regions of the rhizome, using a razor blade. Where possible, samples were taken from nodal regions containing a meristem, the plant tissue from where potential re-growth would occur.

Some of the resultant samples were then saved as control samples, with the remainder being designated as test samples. The control samples were treated with a control acetone formulation, containing no FDA, but otherwise were subjected to the same conditions as the test samples. The test samples were placed in the FDA stain formulation and incubated at room temperature for at least 20 minutes. After incubation, the samples were washed off with distilled water and examined by fluorescence microscopy.

For the FDA fluorescence microscopy test, a Nikon® SMZ 1000 dissection microscope was used with a 100W Nikon® mercury lamp light source, an excitation filter of 480/40 nm, a dichromic mirror of 505 nm and a barrier filter of 535/50 nm. FDA has a maximum absorption and emission at around 490 nm and 520 nm respectively.

The stained test samples were then compared against the control samples and the differences noted. Where a sample was viable (i.e. alive), the cortex region of the rhizome in the test sample was found to fluoresce relative to the corresponding region of a control sample when examined under the microscope. The strongest fluorescence occurred at the edge of the cortex, and faded in smoothly to lesser fluorescence in the inner cortex. Where a sample was not viable (i.e. dead), two different results were noted. Where the sample had died recently, there was no fluorescence in the test sample when compared to the control sample. However, where the sample has been dead for some time, some test samples exhibited a very bright band of fluorescence around the outside of the sample, which could be observed even without the microscope. This band had a constant width and did not fade away. It is believed this band was caused by bacteria in the soil releasing enzymes onto the rhizome to break it down, the ring merely indicating the presence of these enzymes rather than the viability of the rhizome. No other part of the test sample fluoresced.

### Example IV

A resazurin stain formulation was prepared by first preparing a 10 mM stock solution of resazurin in dimethyl sulphoxide (DMSO). This was done by dissolving 0.0025 g resazurin in 1 ml DMSO. From this stock solution, a working solution with a final resazurin concentration of 9.95 μM was prepared by adding 0.1 ml of the stock solution to 100 ml of deionised water. 10 ml of this resazurin stain formulation was used for each sample tested, selected from the rhizomes collected in Example I.

The samples were prepared in the same way as for the FDA assay described in Example III. Again, some of the samples were used as control samples, with the remainder being designated as test samples. Each test sample was placed into 10 ml of the resazurin stain formulation and incubated at room temperature for at least 15 minutes. After incubation, the samples were washed with distilled water and examined using fluorescence microscopy.

For the resazurin fluorescence microscopy test, an Olympus® BH-2 microscope was used, with a 100W Olympus® mercury lamp light source, a green excitation filter (IF-545+PG-36), a green dichromic mirror (DM-580+O-590), and a 610 nm barrier filter. The maximum absorption and emission of resazurin is 563 nm and 587 nm respectively.

The stained test samples were then compared against the control samples and the differences noted. In viable samples, the test sample fluoresced red when viewed under the microscope compared to the control sample. Where the sample was not viable, there was no difference between the test sample and the control sample.

### Example V

A 2,3,5-triphenyltetrazolium chloride (TTC) stain formulation was prepared containing 0.6% (w/v) TTC in 0.05M phosphate buffer at pH 7.4. This was done by mixing 0.18 g of TTC with 0.0204 g potassium phosphate and 0.18 g sodium phosphate. The solution was brought up to 30 ml with deionised water, and the pH adjusted to 7.4 using 10M sodium hydroxide. The resultant TTC stain formulation was then divided into 3 ml aliquots.

As with Examples III and IV, the rhizome samples collected in Example I were prepared by washing the rhizomes thoroughly to remove any soil or other contaminants. Thin slices were then taken of the rhizome (1-2 mm thick) using a razor blade. The layer to the outside of the cambium was then removed as this layer may contain impurities which could alter the result of the experiment. 0.3 g of sampled rhizome material was added to each aliquot, with three separate aliquots being used for each different sampled rhizome.

The resultant test samples were then incubated for 24 hours at 30° C. Once the incubation was complete, the TTC stain formulation was poured away, and each sample was washed three times with deionised water. The colour was then extracted out of each rhizome sample using 4 ml of 95% ethanol at 85° C. for exactly 5 minutes. This was repeated four times, each time saving the extract in a separate tube. The extracts were then brought up to 25 ml with 95% ethanol, and the absorbance at 490 nm was read using a spectrophotometer, against 95% ethanol as a control sample.

The sample was regarded as still viable where the absorbance read by the spectrophotometer was greater than 1.0, but was regarded as not viable where the absorbance was less than 1.0.

The results of the tests carried out in Examples I to V above, are set out in Table 1 below. Table 1 shows results of the tests, verifying the vital stain assessments carried out in Examples III to V, by cross-reference to the culture growth assays carried out in Example II. Rhizome samples numbered from 1-6 were collected from untreated sites (Bath, Bristol) whilst samples 7-10 were collected from sites which had been treated with an anti-knotweed preparation (Shipdham, Aldridge, Chester, Runcorn). The nodal segments of the samples are designated 1-6, this number corresponding to the position of the nodal segment along the rhizome: segment 1 being the most proximal node and segment 6 being the most distal node. The scoring of alive (viable) and dead (non-viable) has been done using the indicators 1 and 0, 1 indicating a positive response and 0 indicating a negative response.

TABLE 1

| Rhizome | Segment | | | | |
|---|---|---|---|---|---|
| sample | number | method tested | alive | dead | Collection point |
| 1 | 2 | FDA | 1 | 0 | Bath |
| 1 | 4 | Resazurin | 1 | 0 | Bath |
| 1 | 6 | TTC | 1 (absorbance reading 1.883) | 0 | Bath |
| 1 | 1 (1.40 g) | culture | 1 | 0 | Bath |
| 1 | 3 (1.28 g) | culture | 1 | 0 | Bath |
| 1 | 5 (1.55 g) | culture | 1 | 0 | Bath |
| Total number of samples | | | 6 | 0 | |
| 2 | 2 | Resazurin | 1 | 0 | Bath |
| 2 | 4 | FDA | 1 | 0 | Bath |
| 2 | 6 | TTC | 1 (absorbance reading 1.187) | 0 | Bath |
| 2 | 1 (2.08 g) | culture | 1 | 0 | Bath |
| 2 | 3 (2.15 g) | culture | 1 | 0 | Bath |
| 2 | 5 (1.31 g) | culture | 1 | 0 | Bath |
| Total number of samples | | | 6 | 0 | |
| 3 | 2 | FDA | 1 | 0 | Bath |
| 3 | 4 | TTC | 1 (absorbance reading 1.961) | 0 | Bath |
| 3 | 5 | Resazurin | 1 | 0 | Bath |
| 3 | 1 (3.28 g) | culture | 1 | 0 | Bath |
| 3 | 3 (2.30 g) | culture | 1 | 0 | Bath |
| 3 | 6 (1.96 g) | culture | 1 | 0 | Bath |
| Total number of samples | | | 6 | 0 | |
| 4 | 1 | FDA | 1 | 0 | Bristol |
| 4 | 3 | Resazurin | 1 | 0 | Bristol |
| 4 | 5 | TTC | 1 (absorbance reading 2.468) | 0 | Bristol |
| 4 | 2 (1.00 g) | culture | 1 | 0 | Bristol |
| 4 | 4 (1.01 g) | culture | 1 | 0 | Bristol |
| 4 | 6 (1.60 g) | culture | 1 | 0 | Bristol |
| Total number of samples | | | 6 | 0 | |
| 5 | 1 | Resazurin | 1 | 0 | Bristol |
| 5 | 3 | FDA | 1 | 0 | Bristol |
| 5 | 5 | TTC | 1 (absorbance reading 1.682) | 0 | Bristol |
| 5 | 2 (1.02 g) | culture | 1 | 0 | Bristol |
| 5 | 4 (1.26 g) | culture | 1 | 0 | Bristol |
| 5 | 6 (1.41 g) | culture | 1 | 0 | Bristol |
| Total number of samples | | | 6 | 0 | |
| 6 | 1 | FDA | 1 | 0 | Bristol |
| 6 | 3 | TTC | 1 (absorbance reading 1.691) | 0 | Bristol |
| 6 | 5 | Resazurin | 1 | 0 | Bristol |
| 6 | 2 (1.91 g) | culture | 1 | 0 | Bristol |
| 6 | 4 (3.75 g) | culture | 1 | 0 | Bristol |
| 6 | 6 (2.83 g) | culture | 1 | 0 | Bristol |
| Total number of samples | | | 6 | 0 | |
| 7 | 2 | FDA | 0 | 1 | Shipdham |
| 7 | 4 | Resazurin | 0 | 1 | Shipdham |
| 7 | 6 | TTC | 1 (absorbance reading 2.056) | 0 | Shipdham |
| 7 | 1 (2.65 g) | culture | 0 | 1 | Shipdham |
| 7 | 3 (1.47 g) | culture | 0 | 1 | Shipdham |
| 7 | 5 (2.14 g) | culture | 0 | 1 | Shipdham |
| Total number of samples | | | 1 | 5 | |
| 8 | 2 | Resazurin | 0 | 1 | Aldridge |
| 8 | 4 | FDA | 0 | 1 | Aldridge |
| 8 | 6 | TTC | 1 (absorbance reading 1.152) | 0 | Aldridge |
| 8 | 1 (1.61 g) | culture | 0 | 1 | Aldridge |
| 8 | 3 (1.74 g) | culture | 0 | 1 | Aldridge |
| 8 | 5 (1.35 g) | culture | 0 | 1 | Aldridge |
| Total number of samples | | | 1 | 5 | |
| 9 | 2 | FDA | 0 | 1 | Chester |
| 9 | 4 | TTC | 0 | 1 | Chester |
| | | | | (0.819) | |
| 9 | 5 | Resazurin | 0 | 1 | Chester |
| 9 | 1 (1.13 g) | culture | 0 | 1 | Chester |
| 9 | 3 (1.27 g) | culture | 0 | 1 | Chester |
| 9 | 6 (1.33 g) | culture | 0 | 1 | Chester |
| Total number of samples | | | 0 | 6 | |
| 10 | 2 | FDA | 0 | 1 | Runcom |
| 10 | 4 | TTC | 0 | 1 | Runcom |
| | | | | (0.456) | |
| 10 | 5 | Resazurin | 0 | 1 | Runcom |
| 10 | 1 (2.00 g) | culture | 0 | 1 | Runcom |
| 10 | 3 (1.69 g) | culture | 0 | 1 | Runcom |
| 10 | 6 (1.74 g) | culture | 0 | 1 | Runcom |

| | | |
|---|---|---|
| Total number of samples | 0 | 6 |

The results of the FDA viability assay agreed completely with the outcome of the culture experiments. All test samples shown to be viable by the culture assay also fluoresced relative to the corresponding control sample when examined under the microscope. The strongest fluorescence was seen at the edge of the cambium, and faded smoothly to centre of the sample. The non-viable test samples did not fluoresce relative to the control samples after incubation with FDA. However, samples 7 and 8, from Shipdham and Aldridge Perry Barr respectively, produced an odd pattern of fluorescence, which was unlike that of the viable samples. It is important to note that in these samples the rhizome had lost its structural integrity. The region to the outside of the cambium was very friable to the touch, and did not fluoresce at all.

For the resazurin viability assay, it was found that where the sample had been determined by the culture assay to be viable, the test sample was seen to fluoresce red when viewed under the microscope, compared to the control sample, which exhibited no fluorescence. For non-viable samples no difference was seen between the stained test sample and the corresponding control sample.

The significant experimental data from the TTC viability assay is the absorbance reading of the extracted formazan. It was found that viable test samples gave an absorbance reading, read by the spectrophotometer, ranging from 1.0 to 3.0. Non-viable (dead) samples gave absorbance readings of below 1.0, and usually around 0.5. However, some samples which had been determined to be non-viable by the culture assay gave an absorbance reading of higher than 1.0. This only occurred in samples which were friable, and corresponded to the samples which produced the odd pattern of fluorescence in the FDA assays. Therefore for these samples the viability needs to be assessed based on an overall comparison including the FDA and resazurin viability assays.

1. A method of determining the viability of a rhizome, comprising the sequential steps of:
(a) sampling material from the rhizome;
(b) contacting material sampled in step (a) with an effective amount of a formulation comprising a first viability indicator;
(c) observing the action of the first viability indicator on the sampled rhizome material, to determine the viability of the rhizome;
(d) contacting material sampled in step (a) with an effective amount of a formulation comprising a further viability indicator different from that used in step (b); and
(e) observing the action of the further viability indicator on the sampled rhizome material, to determine the viability of the rhizome.

2. (canceled)

3. The method as claimed in claim 1, further comprising a plurality of additional sequences (d) and (e), each utilising a different viability indicator.

4. The method as claimed in claim 1, wherein said at least one viability indicator is a vital stain indicator.

5. The method as claimed in claim 4, wherein each viability indicator is a vital stain indicator.

6. The method as claimed in claim 4, wherein each vital stain indicator is selected from the group consisting of fluorescein diacetate, resazurin and tetrazolium chloride salts.

7. The method as claimed in claim 6 wherein the or each vital stain indicator is selected from the group consisting of fluorescein diacetate, resazurin and 2,3,5-triphenyltetrazolium chloride.

8. The method as claimed in claim 6, wherein fluorescein diacetate is selected as a vital stain indicator, said method further including a preliminary step of preparing a fluorescein diacetate stain formulation comprising substantially 0.4 g fluorescein diacetate in 100 ml acetone.

9. The method as claimed in claim 8, wherein in one of steps (b) and (d), material sampled from the rhizome in step (a) is incubated with substantially 5 ml of said fluorescein diacetate stain formulation at room temperature for substantially 20 minutes.

10. The method as claimed in claim 8, wherein in one of steps (c) and (e), the action of the fluorescein diacetate on the sampled rhizome material is observed by fluorescence microscopy.

11. The method as claimed in claim 6, wherein resazurin is selected as a vital stain indicator, said method further including a preliminary step of preparing a resazurin stain formulation as an aqueous solution having a concentration of substantially 9.95 μM.

12. The method as claimed in claim 11, wherein in one of steps (b) and (d), material sampled from the rhizome in step (a) is incubated with substantially 10 ml of resazurin stain formulation at room temperature for at least 15 minutes.

13. The method as claimed in claim 11, wherein in one of steps (c) and (e), the action of the resazurin on the sampled rhizome material is observed by fluorescence microscopy.

14. The method as claimed in claim 6, wherein a tetrazolium chloride salt is selected as a vital stain indicator, said method further including a preliminary step of preparing a tetrazolium chloride salt stain formulation as an aqueous solution having a concentration of substantially 0.6% (w/v) tetrazolium chloride salt and pH of substantially 7.4.

15. The method as claimed in claim 14, wherein in one of steps (b) and (d), 0.3 g of material sampled from the rhizome in step (a) is incubated with substantially 3 ml of tetrazolium chloride salt stain formulation for substantially 24 hours at substantially 30° C.

16. The method as claimed in claim 14, wherein in one of steps (b) and (d), each sample is washed three times with deionised water; and subjected to an extraction process, using substantially 4 ml of 95% ethanol at substantially 85° C. for substantially 5 minutes; said extraction process being repeated four times.

17. The method as claimed in claim 14, wherein in one of steps (c) and (e), the absorbance of the sample is measured at substantially 490 nm using a spectrophotometer.

18. The method as claimed in claim 14, wherein the selected tetrazolium chloride salt is 2,3,5-triphenyltetrazolium chloride.

19. The method as claimed in claim 1, comprising two additional sequences of steps (d) and (e), and wherein each of fluorescein diacetate, resazurin and 2,3,5-triphenyltetrazolium chloride is selected as a viability indicator.

20. The method as claimed in claim 1, wherein step (a) comprises sub-steps of washing the rhizome to remove dirt and any contaminants, and slicing the rhizome into sections of substantially 1-2 mm thickness.

21. The method as claimed in claim 20 wherein in step (a) material is sampled from the rhizome at nodal regions thereof containing a meristem.
